(19) United States
(12) Patent Application Publication
(10) Pub. No.: US 2003/0153904 A1
(43) Pub. Date: Aug. 14, 2003

(54) Environmental chamber for laser refractive surgery

(76) Inventor: Anilbhai S. Patel, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/074,967

(22) Filed: Feb. 13, 2002

(51) Int. Cl. A61B 18/18

(52) U.S. Cl. 606/5

(57) ABSTRACT

An environmental chamber that can be fixed to the eye during laser refractive surgery. The chamber is held in place by a suction ring or the like and contains a window that is transparent to necessary ultraviolet radiation, visible light and infrared radiation wavelengths. The chamber is connected to a control system that controls the temperature and humidity within the chamber and provides for the evacuation of ablation by-products from the surgical site.

### BACKGROUND OF THE INVENTION

This invention relates generally to the field of laser refractive surgery and, more particularly, to a device for controlling the environment at the surgical site during laser refractive surgery.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia), or formed in back of the retina because the eye is abnormally short (axial hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device.

In the past, axial myopia, axial hyperopia and corneal astigmatism generally have been corrected by spectacles or contact lenses, but there are several refractive surgical procedures that have been investigated and used since 1949. Jose Barraquer, M.D. investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). In the 1990s, the use of photablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. Recently, a new version of PRK called Laser Epithelial Keratomileusis (LASEK) has been developed wherein the epithelial layer is non-destructively rolled aside and the underlying stromal tissue is ablated in a manner similar to PRK.

In the past, the amount of tissue removed by the laser was determined by taking pre-operative measurements of the optical errors of the eye, sphere, cylinder and axis, termed “low order” optical aberrations. These measurements were manually loaded into the refractive laser and a proposed corrective “recipe” was calculated by the laser software. More recently, the use of wavefront sensor technology, which measures both the low order optical aberrations and the “higher” order aberrations, such as coma, trefoil and spherical aberration, have been investigated. See for example U.S. Pat. Nos. 5,777,719, 5,949,521, 6,095,651 (Williams, et al.), U.S. patent application Ser. Nos. 09/566,409 and 09/566,668, both filed May 8, 2000, and in PCT Patent Publication No. WO 00/10448, the entire contents of which being incorporated herein by reference. These wavefront sensors are particularly useful when used in combination with a high-speed eye movement tracker, such as the tracker disclosed in U.S. Pat. Nos. 5,442,412 and 5,632,742 (Frey, et al.), the entire contents of which being incorporated herein by reference. The ultimate goal of these devices is to link the wavefront sensor to the laser and eye movement tracker to provide real-time diagnostic data to the laser during surgery. The use of highly accurate diagnostic data along with precision eye movement trackers allows for a very precise, customized surgical procedure.

The excimer lasers used in laser refractive surgery, emitting radiation having a wavelength of approximately 193 or 213 nanometers, have been found to be sensitive to environmental factors such as temperature, humidity and the presence of ablation by-products. Therefore, the surgical outcome can vary depending upon the temperature and humidity of the air in the surgical suite as well as the efficiency of the normal air circulation in removing by-products of the ablation. This variation can compromise the precision of the correction using the available wavefront sensors and eye trackers. Thus, these corneal photorefractive surgical procedures need a device that can precisely control the environmental conditions present at the surgical site during such laser refractive surgery.

One of the issues associated with ablative corneal procedures such as PRK, LASIK and LASEK is the irreversibility of the ablation. Once tissue has been ablated from the corneal stroma, it cannot be replaced, and further touch-up or enhancement procedures are then limited to the remaining thinned cornea. Elimination of this feature of the ablative corneal refractive procedures along with widening of the range of possible correction could have dramatic effects on the future of laser refractive surgery.

A variation of a procedure known as keratophakia is an attempt at doing just that. Rather than ablating the stromal bed of the patient's own cornea, a donor corneal lenticule is ablated. This lenticule is shaped for placement on the stromal bed after the microkeratome-created flap is set aside similar to the LASIK procedure. The surface of the lenticule is then ablated by the excimer laser to the desired shape and covered with the patient's corneal flap. This procedure has the effect of altering the anterior surface of the patient's cornea, thus changing the refractive power of the cornea. In this manner, the patient's corneal stroma is left intact, and correction can be performed on the donor lenticule or by replacement of the donor lenticule. In addition, the range of correction can be broad, as the donor lenticule can be ablated into a variety of shapes. Ablation of the lenticule can provide correction or myopia, hyperopia, astigmatism and even presbyopia by creating a multifocal corneal optic.

Again, however, a significant issue exists. The availability of human donor corneas severely limits this technology from ever capturing a significant share of the laser refractive surgery market. Thus, ablatable, synthetic polymer lenticules are being suggested for use in place of human donor corneal lenticules. See, for example, U.S. Pat. Nos. 5,919,185, 6,063,073 and 6,197,019 (Peyman), the entire contents of which being incorporated herein by reference. One of the requirements of an ablatable, synthetic polymer lenticule is that it should provide the necessary metabolic flux transfer for maintaining the health of the entire cornea, including the overlaying flap, as demonstrated by Jose Barraquer, M.D., and others, for lenticules made from human donor corneal stroma. Suitable intrastromal-safe implant materials are well-documented as being hydrogel materials with water contents of at least 50%. The ablation of such hydrogel materials by excimer laser or any other laser must be very precise, within a few microns, to achieve targeted precision of the refractive correction of vision to with +/−0.25 D or better. The ablation rate of such hydrogel materials is critically dependent on the hydration status of the material which in turn is dependent of the surrounding environmental humidity and temperature. Also, the by-products and debris of the ablation process needs to be safely removed to avoid its undesirable implantation in the corneal stroma. Thus, such ablatable synthetic polymer based keratophakia laser refractive surgical procedures also need a device that can precisely control the environmental conditions present at the surgical site during such laser refractive surgery.

In addition, the controlled removal of ablation by-products for all laser-based refractive surgery may provide increased safety to the patient and to the surgical team present during surgery who might otherwise inhale such by-products.

Accordingly, a need continues to exist for a device that can precisely control the environmental conditions present at the surgical site during laser refractive surgery.

### BRIEF SUMMARY OF THE INVENTION

The present invention improves upon the prior art by providing an environmental chamber that can be fixed to the eye during laser refractive surgery. The chamber is held in place by a suction ring or the like and contains a window that is transparent to necessary ultraviolet radiation, visible light and infrared radiation wavelengths. The chamber is connected to a control system that controls the temperature and humidity within the chamber and provides for the evacuation of ablation by-products from the surgical site.

Accordingly, one objective of the present invention is to provide an environmental chamber that can be fixed to the eye during laser refractive surgery.

Another objective of the present invention is to provide an environmental chamber for use during laser refractive surgery having a control system that controls the temperature and humidity within the chamber.

Another objective of the present invention is to provide a device that controls the temperature and humidity at the surgical site during laser refractive surgery.

These and other advantages and objectives of the present invention will become apparent from the detailed description and claims that follow.

### BRIEF DESCRIPTION OF THE DRAWING

FIG. 1 is a simplified cross-section depiction of the environmental chamber of the present invention.

FIG. 2 is a simplified schematic representation of the environmental chamber system of the present invention.

### DETAILED DESCRIPTION OF THE INVENTION

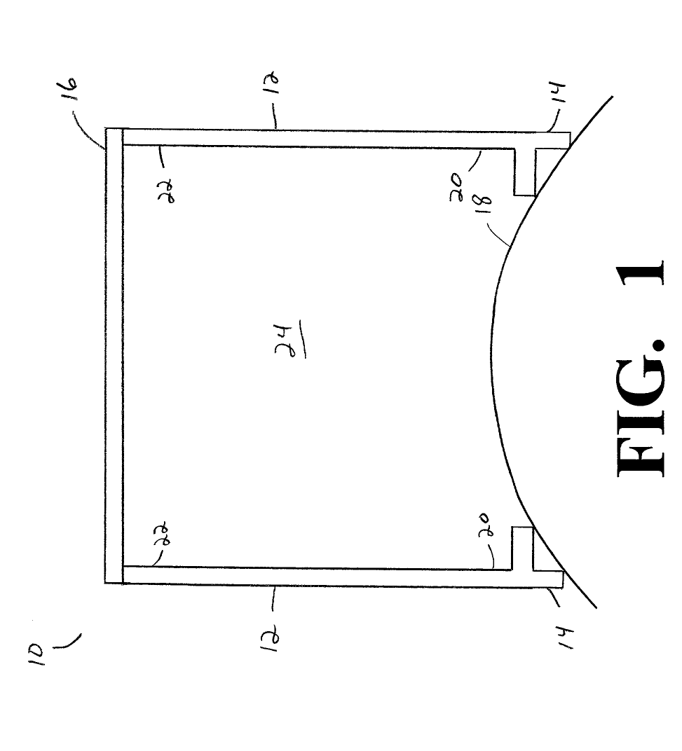

As best seen in FIG. 1, environmental chamber 10 of the present invention generally consists of housing 12, suction ring 14 and ablation window 16. Housing 12 may be generally cylindrical, pyramidal, conical, or any other suitable shape and made from a resterilizable material, such as titanium of stainless steel. Suction ring 14 may be of any desired construction suitable for holding chamber 12 tightly to eye 18. Suitable suction rings 14 being well-known in the art and described, for example, in U.S. Pat. Nos. 4,662,370 (Hoffman, et al.), 5,133,726 (Ruiz, et al.), 5,215,104 (Steinert), 5,496,339 (Koepnick), 5,586,980 (Kremer, st al.), 5,524,456 (Hellenkamp) and 5,643,299 (Bair), the entire contents of which being incorporated herein by reference. Eye 18 and suction ring 14 seal proximal end 20 of housing 12 airtight. Distal end 22 of housing 12 is sealed airtight by window 16. Window 16 is affixed to housing 12 by any suitable, heat and steam-resistant method, such as epoxy or other adhesive, and is preferably made from a material transparent to far and near ultraviolet radiation, as well as visible light and infrared radiation used with eye-tracking devices, such as calcium fluoride, magnesium fluoride, sapphire or any other suitable material. When affixed to eye 18, interior 24 of chamber 10 is sealed from ambient environmental conditions.

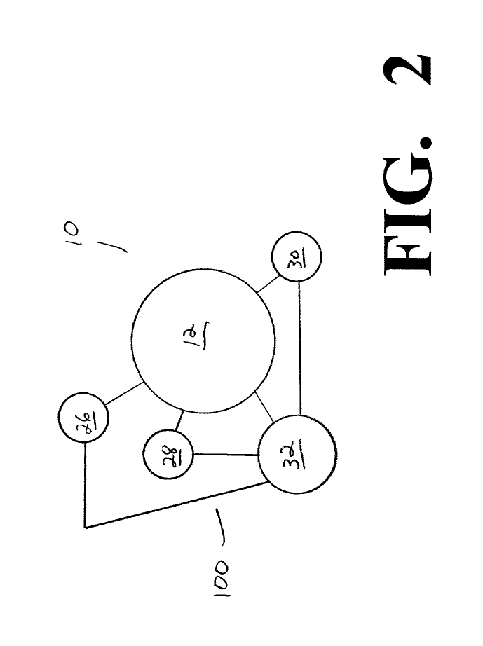

As best seen in FIG. 2, chamber 10 also contains environment control system 100 having temperature sensor 26, humidity sensor 28 and ventilation system 30. Temperature sensor 26, humidity sensor 28 and ventilation system 30 are all in communication with environmental controller 32, and temperature sensor 26, humidity sensor 28, ventilation system 30 and controller 32 all are electronically or fluidly connected to housing 12. Temperature sensor 26 and humidity sensor 28 monitor the humidity and temperature within interior 24 of housing 12 and communicate such information to environmental controller 32. Environmental controller 32 contains suitable heating, cooling and humidification/dehumidification devices so as to maintain the temperature and humidity within interior 24 at any desired level, such devices being well-known in the art. In addition, ventilation system 30 assures that ablation by-products, pair-borne particles, gases and the like, are ventilated from interior 24 so as to not interfere with the action of the refractive laser. Ventilation system 30 communicates with and is under the control of environmental controller 32 so as to maintain the optimum environment within housing 12.

In use, suction ring 14 is placed on eye 18 so as to fixate housing 12 on eye 18. Interior 24 is brought to the proper environmental state by environmental controller 32. The laser refractive surgical procedure can then be completed through window 16 with eye 18 is a sealed and controlled environment.

This description is given for purposes of illustration and explanation. It will be apparent to those skilled in the relevant art that changes and modifications may be made to the invention described above without departing from its scope or spirit.

I claim:

1. A chamber for laser refractive surgery, comprising:
a) a housing having an interior;
b) a suction ring attached to the proximal end of the housing, the suction ring adapted to seal the housing on an eye;
c) a window on the distal end of the housing opposite the suction ring, the window sealing the distal end of the housing; and
d) an environmental control system in communication with the interior, the control system capable of maintaining desired environmental conditions in the interior.

2. The chamber of claim 1 wherein the window is transparent to at least far and near ultraviolet radiation.

3. The chamber of claim 1 wherein the environmental control system comprises an environmental controller capable of maintaining the temperature and humidity within interior at desired levels.

4. The chamber of claim 1 wherein the environmental control system comprises temperature and humidity sensors in communication with an environmental controller capable of maintaining the temperature and humidity within interior at desired levels.

5. The chamber of claim 1 wherein the window comprises calcium fluoride.

6. The chamber of claim 1 wherein the window comprises magnesium fluoride.

7. The chamber of claim 1 wherein the window comprises sapphire.

8. A chamber for laser refractive surgery, comprising:
a) a housing having an interior;
b) a suction ring attached to the proximal end of the housing, the suction ring adapted to seal the housing on an eye;
c) a window made from calcium fluoride or magnesium fluoride or sapphire on the distal end of the housing opposite the suction ring, the window sealing the distal end of the housing; and
d) an environmental control system in communication with the interior, the control system having temperature and humidity sensors and being capable of maintaining desired environmental conditions in the interior.

9. The chamber of claim 8 wherein the environmental control system comprises an environmental controller capable of maintaining the temperature and humidity within interior at desired levels.
